(19) United States
(12) Patent Application Publication
(10) Pub. No.: US 2014/0316452 A1
(43) Pub. Date: Oct. 23, 2014

(54) LIQUID EJECTION DEVICE AND MEDICAL APPARATUS

(71) Applicant: Seiko Epson Corporation, Tokyo (JP)

(72) Inventors: Hirokazu SEKINO, Chino-shi (JP); Hideki KOJIMA, Matsumoto-shi (JP); Kazuaki UCHIDA, Fujimi-machi (JP)

(73) Assignee: SEIKO EPSON CORPORATION, Tokyo (JP)

(21) Appl. No.: 14/255,784

(22) Filed: Apr. 17, 2014

(30) Foreign Application Priority Data
Apr. 18, 2013 (JP) 2013-087313

(51) Int. Cl. A61B 17/3203

(52) U.S. Cl. CPC A61B 17/3203; A61B 2017/00539
USPC 606/167; 222/71

(57) ABSTRACT

A liquid ejection device includes a liquid chamber, a volume varying unit, a liquid supplying unit, and a control unit. An interior volume of the liquid chamber can be varied. The volume varying unit varies the volume in the liquid chamber. The liquid supplying unit supplies liquid to the liquid chamber. The control unit controls the volume varying unit and the liquid supplying unit to thereby adjust the pressure in the liquid chamber.

This application claims the benefit of Japanese Patent Application No. 2013-87313, filed on Apr. 18, 2013. The content of the aforementioned application is incorporated herein by reference in its entirety.

### BACKGROUND

1. Technical Field

The present invention relates to ejection of liquid.

2. Related Art

There is known a liquid ejection device that varies the volume of a liquid chamber using an actuator including a piezoelectric element and generates pulsation in the pressure of the liquid in the liquid chamber to thereby intermittently eject the liquid (e.g., JP-A-2008-82202).

A problem of the related art is that air bubbles tend to accumulate in the liquid chamber. The air bubbles in the liquid chamber are generated from the liquid in the liquid chamber by the volume variation of the liquid chamber. When the air bubbles accumulate in the liquid chamber, the volume variation of the liquid chamber is absorbed by volume variation of the air bubbles. Therefore, appropriate pulsation sometimes cannot be generated.

### SUMMARY

An advantage of some aspects of the invention is to solve at least a part of the problems described above, and the invention can be implemented as the following forms.

(1) An aspect of the invention provides a liquid ejection device that ejects liquid. The liquid ejection device includes: a liquid chamber, an interior volume of which can be varied; a volume varying unit configured to vary the volume in the liquid chamber; a liquid supplying unit configured to supply the liquid to the liquid chamber; and a control unit configured to control the volume varying unit and the liquid supplying unit to thereby adjust the pressure in the liquid chamber. According to this aspect, it is possible to generate appropriate pulsation.

(2) The liquid ejection device of the aspect described above may further include a suction port connected to a suction channel, which communicates with a suction device, and capable of sucking the liquid, and the control section may perform control for setting the pressure higher when the control unit controls the volume varying unit and the liquid supplying unit in a second mode than when the control unit controls the volume varying unit and the liquid supplying unit in a first mode. According to this aspect, in the case of the second mode, since the pressure in the liquid chamber is high compared with the case of the first mode, air bubbles in the liquid chamber are easily discharged through an ejection tube.

(3) In the liquid ejection device of the aspect described above, the control unit may control the liquid supplying unit such that a supply amount of the liquid into the liquid chamber is larger when the control unit performs control in the second mode than when the control unit performs control in the first mode. According to this aspect, in the case of the second mode, since a supply amount of the liquid into the liquid chamber is large compared with the case of the first mode, the air bubbles in the liquid chamber are easily discharged through the ejection tube.

(4) In the liquid ejection device of the aspect described above, the control unit may control the volume varying unit such that variation width of the volume in the liquid chamber is larger when the control unit performs control in the second mode than when the control unit performs control in the first mode. According to this aspect, in the case of the second mode, since the variation width of the volume of the liquid chamber is large compared with the case of the first mode, the air bubbles in the liquid chamber are easily discharged through the ejection tube.

(5) In the liquid ejection device of the aspect described above, the control unit may control the volume varying unit such that the frequency of volume variation of the liquid chamber is larger when the control unit performs control in the second mode than when the control unit performs control in the first mode. According to this aspect, in the case of the second mode, since the frequency of volume variation of the liquid chamber is large compared with the case of the first mode, the air bubbles in the liquid chamber are easily discharged through the ejection tube.

(6) Another aspect of the invention provides a medical apparatus including the liquid ejection device of the aspects described above. According to this aspect, it is possible to provide a medical apparatus including the liquid ejection device in which air bubbles less easily accumulate in the liquid chamber.

Not all of the plurality of components of the aspects of the invention explained above are essential. In order to solve a part or all of the problems explained above or in order to attain a part or all of the effects described in this specification, a part of the plurality of components can be changed, deleted, and replaced with other new components as appropriate and delete apart of limitations of the components. In order to solve a part or all of the problems explained above or in order to attain a part or all of the effects described in this specification, a part or all of the technical features included in one of the aspects of the invention explained above can be combined with a part or all of the technical features included in the other aspects of the invention explained above as an independent aspect of the invention.

For example, one of the aspects of the invention can be implemented as a device including a part or all of the liquid chamber, the volume varying unit, the liquid supplying unit, and the control unit. The device may include or may not include the liquid chamber. The device may or may not include the volume varying unit. The device may or may not include the liquid supplying unit. The device may or may not include the control unit. For example, in the liquid chamber, the interior volume can be varied. For example, the volume varying unit may vary the volume in the liquid chamber. For example, the liquid supplying unit may supply the liquid to the liquid chamber. For example, the control unit may control the volume varying unit and the liquid supplying unit to thereby adjust the pressure in the liquid chamber. Such a device can be implemented as, for example, a liquid ejection device and can be implemented as devices other than the liquid ejection device. According to such a form, it is possible to solve at least one of the various objects such as a reduction in the size of the device, a reduction in costs, resource saving, simplification of manufacturing, and improvement of convenience of use. A part or all of the technical features of the forms of the liquid ejection device explained above can be applied to this device.

The invention can also be implemented in various forms other than the forms explained above. For example, the invention can be implemented in forms such as a liquid ejection method, a surgical operation method, computer programs for implementing these methods, and storage media having these computer programs stored therein.

### BRIEF DESCRIPTION OF THE DRAWINGS

The invention will be described with reference to the accompanying drawings, wherein like numbers reference like elements.

FIG. 1 is a configuration diagram of a liquid ejection device.

FIG. 2 is an internal structure diagram of a hand piece.

FIG. 3 is a flowchart for explaining startup processing.

### DESCRIPTION OF EXEMPLARY EMBODIMENTS

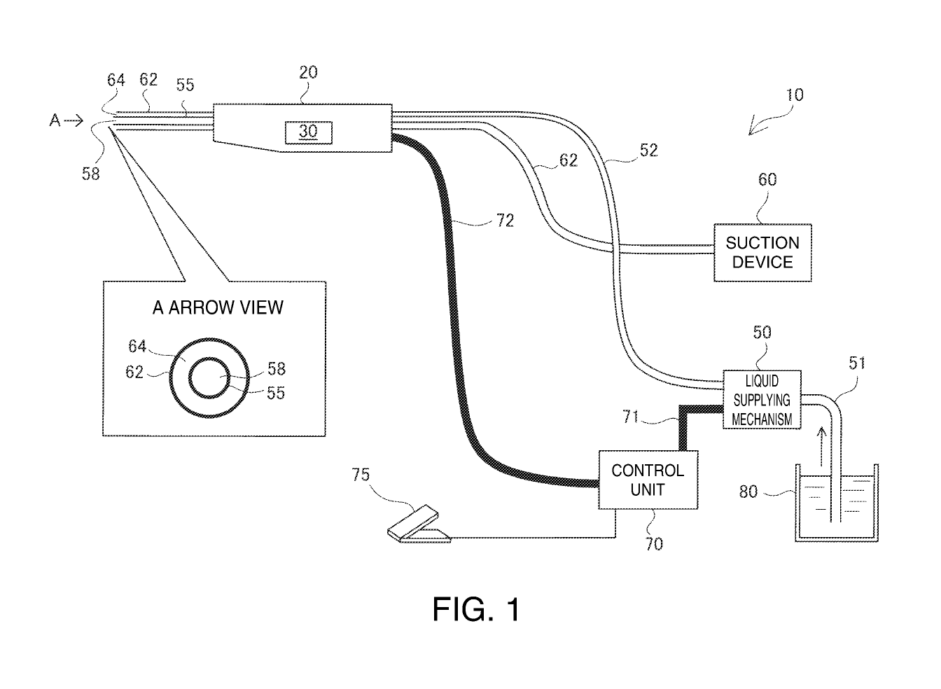

FIG. 1 shows the configuration of a liquid ejection device 10. The liquid ejection device 10 is a medical device used in a medical institution. The liquid ejection device 10 has a function of incising or excising an affected part by ejecting liquid to the affected part.

The liquid ejection device 10 includes a hand piece 20, a liquid supplying mechanism 50, a suction device 60, a control unit 70, and a liquid container 80. The liquid supplying mechanism 50 and the liquid container 80 are connected to each other by a connection tube 51. The liquid supplying mechanism 50 and the hand piece 20 are connected to each other by a liquid supplying channel 52. The connection tube 51 and the liquid supplying channel 52 are formed of resin. The connection tube 51 and the liquid supplying channel 52 may be formed of a material other than the resin.

The liquid container 80 stores saline. The liquid supplying mechanism 50 supplies liquid sucked from the liquid container 80 via the connection tube 51 to the hand piece 20 via the liquid supplying channel 52.

The hand piece 20 is an instrument held and operated by a user of the liquid ejection device 10. A pulsation generating unit 30 incorporated in the hand piece 20 generates pulsation in the pressure of the liquid supplied to the hand piece 20 from the liquid supplying mechanism 50 via the liquid supplying channel 52. The liquid, in which the pulsation in the pressure is generated, is supplied to an ejection tube 55. The liquid supplied to the ejection tube 55 is intermittently ejected from an ejection port 58. The user hits the liquid ejected from the ejection port 58 on an affected part to thereby incise or excise the affected part. The ejection tube 55 is formed of stainless steel. The ejection tube 55 may be formed of other materials having predetermined or higher rigidity such as other kinds of metal such as brass and reinforced plastics.

The control unit 70 transmits a drive signal to the pulsation generating unit 30 via a signal cable 72. The control unit 70 controls the liquid supplying mechanism 50 via a control cable 71 to thereby control a flow rate of the liquid supplied to the pulsation generating unit 30. A foot switch 75 is connected to the control unit 70. When the user turns on the foot switch 75, the control unit 70 controls the liquid supplying mechanism 50 to execute the supply of the liquid to the pulsation generating unit 30. The control unit 70 transmits the drive signal to the pulsation generating unit 30 and causes the pulsation generating unit 30 to generate pulsation in the pressure of the liquid supplied to the pulsation generating unit 30.

The suction device 60 is a device for sucking the liquid and an excised object around the ejection port 58. The suction device 60 and the hand piece 20 are connected to each other by a suction channel 62. The suction channel 62 pierces through the hand piece 20 and opens near the distal end of the ejection tube 55. The suction channel 62 covers the ejection tube 55 in the hand piece 20 to thereby forma cylinder in which the wall of the ejection tube 55 and the wall of the suction channel 62 are substantially concentric as shown in an A arrow view of FIG. 1. A channel through which a sucked object sucked from a suction port 64, which is the distal end of the suction channel 62, flows is formed between the outer wall of the ejection tube 55 and the inner wall of the suction channel 62. The sucked object is sucked by the suction device 60 via the suction channel 62.

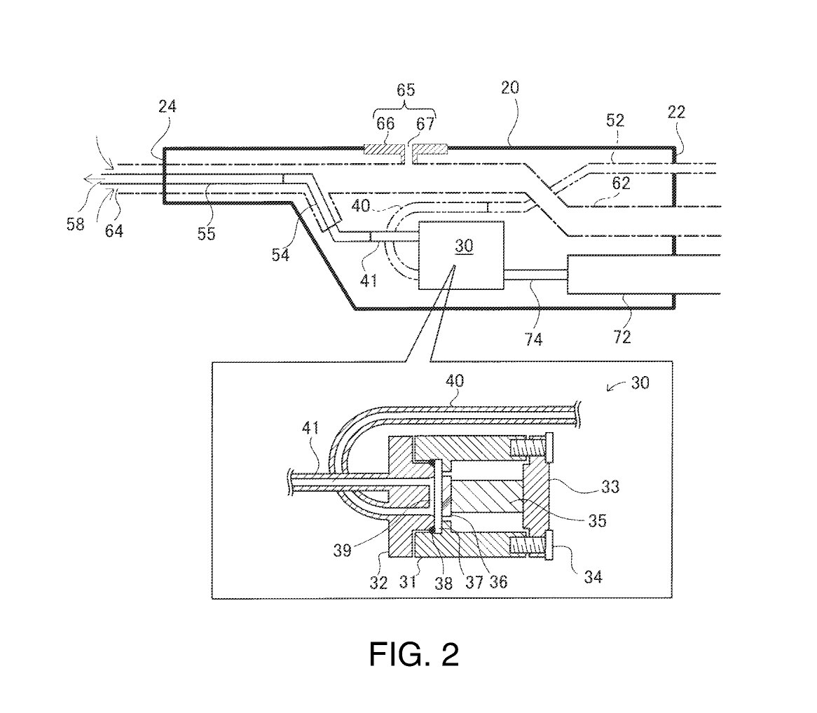

FIG. 2 shows the internal structure of the hand piece 20. The hand piece 20 incorporates a pulsation generating unit 30, an inlet channel 40, an outlet channel 41, and a connection tube 54 and includes a suction force adjusting mechanism 65.

The pulsation generating unit 30 includes, as shown in the lower part of FIG. 2, a first case 31, a second case 32, a third case 33, bolts 34, a piezoelectric element 35, a reinforcing plate 36, a diaphragm 37, a gasket 38, the inlet channel 40, and the outlet channel 41. The first case 31 and the second case 32 are opposed and joined to each other. The first case 31 is a cylindrical member. One end portion of the first case 31 is closed by fixing the third case 33 with the bolts 34. The piezoelectric element 35 is arranged in a space formed on the inside of the first case 31.

The piezoelectric element 35 is a laminated piezoelectric element. One end of the piezoelectric element 35 is fixedly attached to the diaphragm 37 via the reinforcing plate 36. The other end of the piezoelectric element 35 is fixedly attached to the third case 33. The diaphragm 37 is made of a metal thin film. The peripheral edge portion of the diaphragm 37 is fixedly attached to the first case 31. A liquid chamber 39 is formed between the diaphragm 37 and the second case 32. The volume of the liquid chamber 39 is varied by the driving of the piezoelectric element 35.

The signal cable 72 is inserted from a rear end portion 22 of the hand piece 20. Two electrode lines 74 are housed in the signal cable 72 and connected to the piezoelectric element 35 in the pulsation generating unit 30. The drive signal transmitted from the control unit 70 is transmitted to the piezoelectric element 35 via the electrode lines 74 in the signal cable 72. The piezoelectric element 35 expands and contracts on the basis of the drive signal.

The inlet channel 40, into which the liquid flows, is connected to the second case 32. The inlet channel 40 is bent in a U shape and extends toward the rear end portion 22 of the hand piece 20. The liquid supplying channel 52 is connected to the inlet channel 40. The liquid supplied from the liquid supplying mechanism 50 is supplied to the liquid chamber 39 via the liquid supplying channel 52.

The piezoelectric element 35 expands and contracts at a predetermined frequency, the diaphragm 37 vibrates. When the diaphragm 37 vibrates, the volume of the liquid chamber 39 varies and the pressure of the liquid in the liquid chamber 39 pulsates. The liquid passed through the liquid chamber 39 flows out from the outlet channel 41.

The outlet channel 41 is connected to the second case 32. The ejection tube 55 is connected to the outlet channel 41 via the connection tube 54. The liquid flowed out to the outlet channel 41 is ejected from the ejection port 58 through the connection tube 54 and the ejection tube 55.

On the other hand, the suction force adjusting mechanism 65 is a mechanism for adjusting a force of the suction channel 62 for sucking the liquid or the like from the suction port 64. The suction force adjusting mechanism 65 includes an operation unit 66 and a hole 67. The hole 67 is a through-hole that connects the suction channel 62 and the operation unit 66. When the user opens and closes the hole 67 with a finger of the hand that grips the hand piece 20, an amount of the air flowing into the suction channel 62 via the hole 67 is adjusted according to a degree of the opening and closing. Consequently, a suction force of the suction port 64 is adjusted. The adjustment of the suction force can also be implemented by control by the suction device 60.

The hole 67 is preferably faced upward in the vertical direction because a sucked object is discharged to the outside from the hole 67. This phenomenon guides the user to grip the hand piece 20 in a posture in which the hole 67 is faced upward in the vertical direction. In the following explanation, the axial direction of the hole 67 is defined as “up down direction”. A direction from a connecting place of the hole 67 and the suction channel 62 to an opening section of the hole 67 is defined as “upward in the up down direction”. In the following explanation, when “upward” is simply referred to, this indicates upward in the up down direction.

In the liquid ejection device 10, when the hole 67 is faced upward, the positions of the components are determined to make the function and operability of the suction force adjusting mechanism 65 preferable. However, the suction force adjusting mechanism 65 does not force the user to adopt a certain method of use of the hand piece 20.

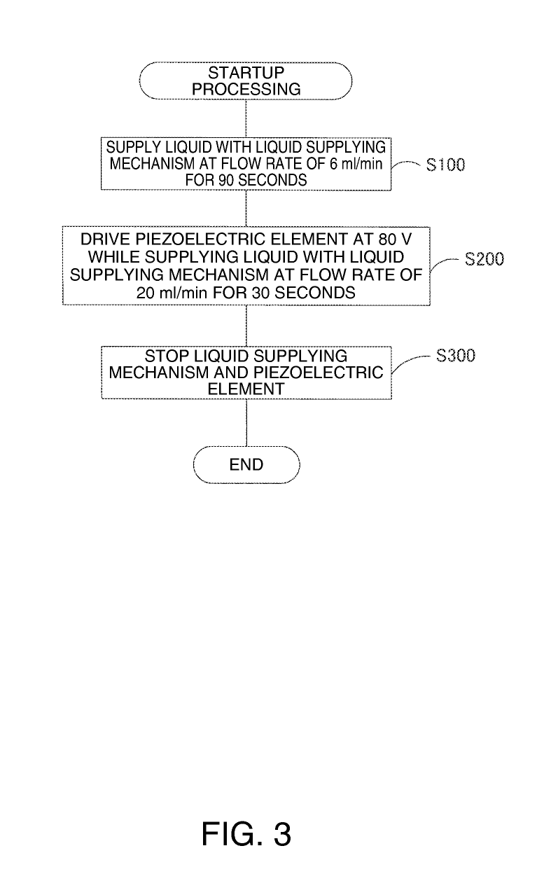

FIG. 3 is a flowchart for explaining startup processing. The startup processing is executed by the control unit 70 when startup of the liquid ejection device 10 is instructed via an input interface provided in the control unit 70.

First, the control unit 70 controls the liquid supplying mechanism 50 at a flow rate of 6 ml/min for 90 seconds and carries out liquid supply (step S100). When a predetermined time elapses from the start of step S100, the liquid starts to be ejected from the ejection port 58. Step S100 is executed for the purpose of filing the liquid in the entire channel through which the liquid flows, in particular, the liquid chamber 39.

Subsequently, the control unit 70 applies 80 V to the piezoelectric element 35 at a predetermined frequency (e.g., 500 Hz) while carrying out the liquid supply by the liquid supplying mechanism 50 at a flow rate of 20 ml/min for 30 seconds (step S200). Thereafter, the control unit 70 stops the liquid supplying mechanism 50 and the piezoelectric element 35 (step S300). Step S200 is executed for the purpose of degassing the entire channel, in particular, the liquid chamber 39. The degassing means discharging air bubbles mixed in the liquid. The air bubbles could also be held up in the liquid chamber 39 by the filling of the liquid by the liquid supplying mechanism 50.

The flow rate of 20 ml/min is set as a value as high as possible in a range in which the liquid can be safely supplied. The flow rate is higher than a value in a use mode (e.g., 6 ml/min). The use mode is a mode for ejecting the liquid for excision and the like of an affected part after the startup processing. On the other hand, an operation mode in step S200 is referred to as degassing mode in this embodiment.

As a condition for the degassing mode, a value and a frequency of a drive voltage applied to the piezoelectric element 35 are set as values as high as possible in a range in which a safe operation is possible, and the value and the frequency are larger than values in the use mode. When any of the flow rate of the liquid and the drive voltage and the drive frequency of the piezoelectric element 35 is high, the degassing of the liquid chamber 39 is facilitated.

For example, a mechanism for facilitating the degassing is estimated as explained below. When the flow rate increases, flow velocity increases and a Reynolds' number increases. When the Reynolds' number increases, a turbulent flow tends to occur. Compared with a laminar flow, the turbulent flow has a large effect of discharging air bubbles held up at a corner of a channel or the like. Therefore, when the flow rate increases, the degassing is considered to be facilitated.

On the other hand, the increase in the flow rate and the increase in the drive voltage of the piezoelectric element 35 increase the pressure in the liquid chamber 39. When the pressure in the liquid chamber 39 increases, air bubbles tend to dissolve in the liquid. The dissolved air bubbles tend to be discharged from the liquid chamber 39 together with the liquid. Under the conditions in step S200, the pressure in the liquid chamber 39 reaches, for example, 0.5 MPa.

When the drive voltage of the piezoelectric element 35 further increases, variation of flow conditions in the liquid chamber 39 increases. The air bubbles are sometimes held up in the liquid chamber 39 by an eddy or stagnation that occurs under predetermined flow conditions. On the other hand, when the flow conditions greatly vary, it is highly likely that the eddy or the stagnation disappears. Therefore, when the drive voltage of the piezoelectric element 35 is increased, the degassing of the liquid chamber 39 is facilitated.

The increase in the drive frequency of the piezoelectric element 35 leads to an increase in the number of times the effects are displayed in a unit time. Therefore, the degassing of the liquid chamber 39 is facilitated.

All the mechanisms concerning the degassing are only estimations. This embodiment is not limited to the facilitation of the degassing by the mechanisms.

After executing the startup processing, when the foot switch 75 is turned on, the control unit 70 intermittently ejects the liquid according to conditions in the use mode. After executing the startup processing, when receiving an instruction for the degassing via the input interface, the control unit 70 executes step S200 and step S300 in the startup processing as operation in the degassing mode. According to the execution of the steps, removal of the air bubbles held up in the liquid chamber 39 and the like in the use mode is realized.

It is preferable that the degassing mode is executed in a state in which the ejection port 58 is put in the liquid. This is because it is possible to visually check whether the air bubbles are discharged from the ejection port 58.

The piezoelectric element 35 and the diaphragm 37 in this embodiment are equivalent to a volume varying unit in the appended claims.

The invention is not limited to the embodiments, examples, and modifications described in this specification and can be implemented in various configurations without departing from the spirit of the invention. For example, technical features of the embodiments, the examples, and the modifications corresponding to technical features in the forms described in the summary of the invention can be replaced or combined as appropriate in order to solve a part or all of the problems explained above or attain a part or all of the effects explained above. Unless the technical features are not explained in this specification as essential technical features, the technical features can be deleted as appropriate. Technical features explained below are examples of such technical features.

The conditions in the degassing mode may be changed. The flow rate may be, for example, 6 to 30 ml/min. The drive voltage may be 50 to 100 V. The time may be 20 to 40 seconds. The drive frequency may be 300 to 1000 Hz. All the parameters may be values other than the values explained above.

The liquid ejection device may be used in apparatuses other than the medical apparatus. For example, the liquid ejection device may be used in a cleaning apparatus that removes stains using ejected liquid. The liquid ejection device may be used in a rendering apparatus that draws a line or the like using ejected liquid.

What is claimed is:

1. A liquid ejection device that ejects liquid, the liquid ejection device comprising:
a liquid chamber, an interior volume of which can be varied;
a volume varying unit configured to vary the volume in the liquid chamber;
a liquid supplying unit configured to supply the liquid to the liquid chamber; and
a control unit configured to control the volume varying unit and the liquid supplying unit to thereby adjust pressure in the liquid chamber.

2. The liquid ejection device according to claim 1, further comprising a suction port connected to a suction channel, which communicates with a suction device, and capable of sucking the liquid, wherein
the control section performs control for setting the pressure higher when the control unit controls the volume varying unit and the liquid supplying unit in a second mode than when the control unit controls the volume varying unit and the liquid supplying unit in a first mode.

3. The liquid ejection device according to claim 2, wherein the control unit controls the liquid supplying unit such that a supply amount of the liquid into the liquid chamber is larger when the control unit performs control in the second mode than when the control unit performs control in the first mode.

4. The liquid ejection device according to claim 2, wherein the control unit controls the volume varying unit such that variation width of the volume in the liquid chamber is larger when the control unit performs control in the second mode than when the control unit performs control in the first mode.

5. The liquid ejection device according to claim 2, wherein the control unit controls the volume varying unit such that the frequency of volume variation of the liquid chamber is larger when the control unit performs control in the second mode than when the control unit performs control in the first mode.

6. A medical apparatus comprising the liquid ejection device according to claim 1.

7. A medical apparatus comprising the liquid ejection device according to claim 2.

8. A medical apparatus comprising the liquid ejection device according to claim 3.

9. A medical apparatus comprising the liquid ejection device according to claim 4.

10. A medical apparatus comprising the liquid ejection device according to claim 5.
